(19) United States
(12) Patent Application Publication
(10) Pub. No.: US 2016/0045762 A1
(43) Pub. Date: Feb. 18, 2016

(54) DISPLACEMENT-BASED CONTROL OF A SKIN TREATMENT DEVICE

(71) Applicants: Martin GUROVICH, Tel Aviv (IL); Josef LUZON, Bet Yehoshua (IL); DERMA DREAM GROUP LTD., Tortola (VG)

(72) Inventors: Martin GUROVICH, Tel Aviv (IL); Josef LUZON, Bet - Yehoshua (IL)

(21) Appl. No.: 14/778,702

(22) PCT Filed: Mar. 20, 2014
(86) PCT No.: PCT/IL2014/050305

§ 371 Date: Sep. 21, 2015

Related U.S. Application Data
(60) Provisional application No. 61/803,959, filed on Mar. 21, 2013.

(51) Int. Cl. A61N 5/06

(52) U.S. Cl. CPC A61N 5/0616; A61N 2005/0626; A61N 2005/0644; A61N 2005/0642; A61N 2005/0651; A61N 2005/0654; A61N 2005/067

(57) ABSTRACT

A device for treating a skin surface includes a treatment applicator to apply a treatment to a region of the skin surface on which the treatment applicator is placed. A displacement sensor senses a displacement of the device relative to the skin surface when the a treatment applicator is placed on the skin surface. A controller controls application of the treatment in accordance with the sensed displacement.

### FIELD OF THE INVENTION

The present invention relates to skin treatment devices. More particularly, the present invention relates to displacement-based control of a skin treatment device.

### BACKGROUND OF THE INVENTION

During recent decades, phototherapy (also known as light-based therapy) has become widely used in the field of dermatology and skin care. Various phototherapy technologies and techniques exist. The technologies and techniques differ from one another with respect to the type of light source and biological function or mechanism involved. Some phototherapy techniques apply selective photothermolysis for the purpose of hair removal, removal of capillary lesions, or tattoo removal. Phototherapy techniques may be applied to achieve skin rejuvenation by collagen stimulation. A laser or radiofrequency source may be applied to achieve skin ablation. Other skin treatments, not based on phototherapy, are also in widespread use. Such non-light-based skin treatments include electrothermolysis for pain relief, and wrinkle and cellulite treatment based on electromagnetic subcutaneous heating, increasing cell metabolism rates, increasing blood flow, causing adipocyte necrosis (fat cell shrinkage), or stimulating collagen remodeling. Mechanical massage is used in countering, squeezing, or traumatizing subcutaneous fat, and in stimulating the vascular system and lymphatic drainage.

Phototherapy devices such as intense pulsed light (IPL) or lasers may be designed to target particular chromophores in the skin. Such devices may be applied to treat various cosmetic or dermatological conditions or concerns. The conditions or treatments may include acne, photo-rejuvenation, telangiectasia, rhytids, dyschromia (e.g., freckling, dark spots, sun damage, capillaries, pigmented and vascular lesions, rosacea, or other blemishes). Such treatment typically has little or no effect on unaffected skin. In some instances IPL may be used for hair removal. Due to the interaction of such treatments with different chromophores in the skin, some of the treatment parameters may be adjusted according to characteristics of the patient's skin, such as skin tone.

Such phototherapy and other treatments often require a full coverage of a target area. On the other hand, overlapping of treated regions of the area is to be avoided. Repetitive treatment of a single region, either by overlapping of treated regions or by unintentional repeated application of a previously treated region could cause injury to the skin. A user of a device for such skin treatments, whether the device is for professional or for home use, is expected to manipulate the device to cover the area to be treated of the treated area while avoiding repeated application of the treatment to a single region of the area.

### SUMMARY OF THE INVENTION

There is thus provided, in accordance with some embodiments of the present invention, a device for treating a skin surface, the device including: a treatment applicator to apply a treatment to a region of the skin surface on which the treatment applicator is placed; a displacement sensor to sense a displacement of the device relative to the skin surface when the treatment applicator is placed on the skin surface; and a controller to control application of the treatment in accordance with the sensed displacement.

Furthermore, in accordance with some embodiments of the present invention, the treatment applicator includes a radiation source to irradiate the region.

Furthermore, in accordance with some embodiments of the present invention, radiation source includes a light emitting diode, a laser, or a xenon lamp.

Furthermore, in accordance with some embodiments of the present invention, an intensity of the radiation is controllable in accordance with the displacement.

Furthermore, in accordance with some embodiments of the present invention, the controller is configured to control application of the treatment avoid excessive application of the treatment to a single region of the skin surface.

Furthermore, in accordance with some embodiments of the present invention, the device further includes an alert device.

Furthermore, in accordance with some embodiments of the present invention, the controller is configured to control the alert device to indicate a comparison of a rate of the sensed displacement with a correct rate of displacement.

Furthermore, in accordance with some embodiments of the present invention, the device is handheld and self-contained.

Furthermore, in accordance with some embodiments of the present invention, the displacement sensor includes an optical sensor that is configured to acquire a sequence of images of the skin surface.

Furthermore, in accordance with some embodiments of the present invention, the displacement sensor includes a ball that is configured to rotate when placed on the skin surface and displaced along the skin surface.

There is further provided, in accordance with some embodiments of the present invention, a method to control a device for treating a skin surface, the method including: obtaining a sensed displacement of the treatment head relative to the skin surface when the treatment head is placed on the skin surface; and controlling operation of a treatment applicator to apply a treatment to the skin in accordance with the sensed displacement.

Furthermore, in accordance with some embodiments of the present invention, controlling the operation includes calculating a current position of the treatment head on the skin surface.

Furthermore, in accordance with some embodiments of the present invention, controlling the operation includes modifying application of the treatment when the current position is within a predetermined distance from a position at which the treatment was previously applied.

Furthermore, in accordance with some embodiments of the present invention, controlling the operation includes calculating a rate of displacement of the treatment head.

Furthermore, in accordance with some embodiments of the present invention, controlling the operation includes adjusting application of the treatment to correspond to the calculated rate of displacement.

Furthermore, in accordance with some embodiments of the present invention, the method further includes comparing the calculated rate of displacement with a correct rate of displacement.

Furthermore, in accordance with some embodiments of the present invention, the method further includes generating an alert indicative of the result of the comparing of the calculated rate with the correct rate.

Furthermore, in accordance with some embodiments of the present invention, the method further includes sensing a characteristic of the skin surfaceand controlling operation of the treatment applicator to apply the treatment in accordance with the sensed characteristic.

Furthermore, in accordance with some embodiments of the present invention, the method further includes sensing a proximity of the skin surface and controlling operation of the treatment applicator to apply the treatment in accordance with the sensed proximity.

Furthermore, in accordance with some embodiments of the present invention, application of the treatment includes irradiating the skin surface.

### BRIEF DESCRIPTION OF THE DRAWINGS

In order to better understand the present invention, and appreciate its practical applications, the following Figures are provided and referenced hereafter. It should be noted that the Figures are given as examples only and in no way limit the scope of the invention. Like components are denoted by like reference numerals.

FIG. 1 is a schematic illustration of a skin treatment device, in accordance with an embodiment of the present invention.

FIG. 2A is a schematic illustration of a mechanical displacement sensor for a skin treatment device, in accordance with an embodiment of the present invention.

FIG. 2B is a schematic illustration of an optical displacement sensor for a skin treatment device, in accordance with an embodiment of the present invention.

FIG. 3 is a flowchart depicting a method of operation of a skin treatment device in accordance with an embodiment of the present invention.

### DETAILED DESCRIPTION OF THE INVENTION

In the following detailed description, numerous specific details are set forth in order to provide a thorough understanding of the invention. However, it will be understood by those of ordinary skill in the art that the invention may be practiced without these specific details. In other instances, well-known methods, procedures, components, modules, units and/or circuits have not been described in detail so as not to obscure the invention.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium (e.g., a memory) that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently.

In accordance with embodiments of the present invention, a skin treatment device includes a treatment applicator that is configured to apply a treatment to a skin surface when the treatment applicator is placed on the skin surface. A treatment head that includes the treatment applicator further includes a displacement sensor. The displacement sensor is configured to sense or measure a displacement relative to the surface. The sensed displacement may be used to determine at least a relative current position of the treatment head on the skin surface.

A section of the skin treatment device that is configured for placement on the skin surface during application of the treatment is herein referred to as the treatment head. The treatment head incorporates at least the treatment applicator and the displacement sensor. The treatment head may include other components. Other components may include, for example, a proximity or contact sensor, a sensors relevant to a particular type of treatment (e.g., for sensing a characteristic or property of the skin that is indicative of a requirement for treatment or a result of treatment), or another sensor or component. The treatment head may or may not be detachable from the remainder of the skin treatment device.

The treatment head (including the treatment applicator and displacement sensor) is herein referred to as being placed on the skin surface when the treatment head is sufficiently close to the skin surface to enable application of the treatment to the skin and operation of the displacement sensor. Being placed on the surface may not require physical contact with the skin surface. In some cases, the treatment head is considered to be placed on the skin surface when the distance between the treatment head and the skin surface is less than 1 cm. In some cases, the treatment head is considered to be placed on the skin surface when the distance between the treatment head and the skin surface is less than 5 mm. In some cases, the treatment head is considered to be placed on the skin surface when the distance between the treatment head and the skin surface is less than 2 mm. In some cases, the treatment head is considered to be placed on the skin surface when the distance between the treatment head and the skin surface is less than 1 mm.

A user of the device places a treatment head of the device on a skin surface to be treated. While the treatment head is in placed on the skin surface, a treatment applicator applies the treatment to the skin surface. For example, the treatment applicator may include a radiation source, with the treatment including irradiation of the skin surface. In this case, the treatment applicator may include a radiation source such as a light emitting diode (LED) or a laser light source. Other examples of treatments may include application of heat, an electromagnetic field, a mechanical treatment (e.g., mechanical massage, vibrator, shaver, or other mechanical treatment), a substance (e.g., vapor, liquid, cream, gel, powder, or other substance), or other treatments.

The skin treatment device may be handheld and self contained. As used herein, a device is referred to as being a handheld device when the device may be held in one or two hands when in operation. As used herein, a handheld device is referred to as being a self-contained device when the device requires no connections to other devices to operate, with the possible exception of a connection to an external power supply (e.g., electrical mains or external power transformer or converter).

A displacement sensor detects displacement of the treatment head relative to the skin surface. For example, the displacement sensor may include an optical or a mechanical component that is configured to measure a displacement relative to a surface.

A mechanical displacement sensor may include a two-dimensionally rotatable mechanical element (e.g., a ball) that may be placed in contact with the skin surface. When the treatment head and mechanical displacement sensor are moved relative to the skin surface, the rotatable mechanical element is rotated. Rotation of the rotatable mechanical element may, in turn, rotate two or more rollers or shafts that are arranged along different axes. Each roller is provided with an encoder (e.g., that includes a chopper wheel) that produces an electrical signal that is indicative of the rotation of the roller. A processor is configured to interpret rotation of the rollers as movement of the two-dimensionally rotatable element relative to the skin surface. Operation of the mechanical sensor may thus be similar to operation of a mechanical computer mouse.

An optical displacement sensor may include a light source (e.g., a solid state light source such as a LED or diode laser, a xenon lamp, or other light source) and camera (e.g., which includes a solid state sensor array and focusing and light collecting optics such as a lens or mirror, and which may include other optical components such as apertures or filters). When the optical sensor is placed on the skin surface, the surface is illuminated by the light source and the illuminated surface is imaged by the camera. A processor is configured to compare sequentially acquired images or frames of the illuminated surface and to detect any shift. A shift of the image is interpreted as a displacement (direction and magnitude) of the sensor relative to the surface. Operation of the optical sensor may thus be similar to operation of an optical computer mouse.

Use of an optical displacement sensor instead of a mechanical displacement sensor may be indicated where there is a risk that moving parts of a mechanical displacement sensor may become clogged. For example, moving parts may become clogged where topical serums or creams are used during skin treatment, by dirt, loose skin particles, or perspiration that occur on the skin surface, by hairs extending from the skin surface, or by applied or naturally occurring materials or objects on the skin surface.

An optical displacement detector may be specifically configured for measurement of displacement relative to human skin. For example, a light source, optical filter, or lens of the optical displacement detector may be selected such that the acquired images contain sufficient contrast between microscopic textural features and the background of the skin. For example, certain wavelengths may be specifically selected to increase the contrast between hair or capillaries and the skin.

Application of the treatment to the skin by the treatment applicator may be controlled in accordance with the sensed displacement. For example, a controller of the treatment applicator may be configured to receive a signal from the displacement sensor that is indicative of the sensed displacement.

The controller may be configured to control the treatment applicator in accordance with the sensed displacement. The controller may be configured to enable, disable, or modify application of the treatment in accordance with the sensed displacement. For example, after the treatment is applied to a region of the skin surface for a predetermined period of time, the controller may disable further application of the treatment until a predetermined threshold displacement is sensed. The threshold displacement may eliminate any overlap between treated regions, or may allow some overlap. As another example, the controller may control a rate of application of the treatment in accordance with a sensed rate of movement of the treatment head over the skin surface. The controller may control an intensity of radiation that is radiated by a radiation source, a rate of heating, or otherwise control an energy, intensity, or rate of application of a treatment.

The sensed displacement may be used to calculate a position on the skin surface (e.g., relative to an initial position). The controller may be configured to prevent repeated application of the treatment to a position that had previously been treated. For example, if the current position is displaced by less than a predetermined threshold distance from a previously treated position, application of the treatment may completely or partially interrupted. Treatment may be resumed when the treatment head is displaced to beyond the threshold distance.

The controller may be configured to operate an alert. The alert may be indicative of application of treatment, or of a change or cessation of application of treatment. The alert may be indicative of a correct rate of displacement of the treatment head over the skin surface, or of a deviation from a correct rate. As used herein, a correct rate of displacement refers to a required, desired, or recommended range of displacement rates. For example, the treatment device may include one or more alarm or alert systems that may be operated by the controller to indicate a status to the user. An alert system may be configured to produce one or more of an audible, visible, or palpable indication. The produced indication may be indicative of operation of the device, of current application of the treatment, of temporary or permanent (e.g., until operation or a reset or restart control by the user) cessation of application of the treatment, that treatment is enabled (e.g., after sensing displacement to an untreated region of the skin surface), of a deviation of a sensed rate of displacement from a correct rate of displacement, displacement at a correct rate of displacement, or any combination of the above.

For example, an audible alert may include a bell, beeper, buzzer, clicker, siren, whistle, tone generator, or other component that is configured to produce a sound. A produced sound (e.g., with a particular tone, volume, beep rate or duration, modulation, tone pattern, or other characteristic of the produced sound) may be indicative of displacement that is slower than, approximately equal to, or greater than the correct rate of displacement. A visible alert may include a light, set of lights, or display that may be configured to produce a visible indication. A particular visible indication (e.g., location or color of a light, number of lights lit, rate of flashing or blinking, displayed content) may be indicative of displacement that is slower than, approximately equal to, or greater than the correct rate of displacement. A palpable or tactile alert may include a vibrator that is configured to produce a particular type or pattern of vibration that is indicative of displacement that is slower than, approximately equal to, or greater than the correct rate of displacement.

Dermatological or cosmetic treatment using a skin treatment device in accordance with an embodiment of the present invention may be advantageous. Automatic control of application of treatment in accordance with sensed displacement may enable effective operation by a user who is displacing the treatment head in a manner that is natural or comfortable to that user. In some cases, indication of a correct displacement rate may guide a user in moving the treatment head at an optimum rate, or assist in training an inexperienced user in correctly displacing the treatment head. Automatic prevention of repeated application of a treatment to a single region of the skin removes from the user the necessity to remember which regions were already treated and which were not. Automatic guidance with regard to displacement of the treatment head may enable efficient (e.g., with regard to coverage in a period of time) and complete coverage of regions to be treated.

In accordance with some embodiments of the present invention, components of an optical displacement sensor may be utilized to sense or measure characteristics of the skin surface. For example, acquired images of the skin surface may be analyzed to detect one or more features of the skin surface that are relevant to the applied treatment. For example, a detected feature or characteristic of the skin surface may include pigmentation, skin tone, uniformity or evenness, redness, capillaries, veins, dark spots, beauty spots, or other features. The optical displacement sensor may be provided with a variety of light sources, each configured to enhance detection of different types of features or characteristics. The detected features or characteristics may be utilized in controlling application of the treatment. For example, a controller of the skin therapy device may adjust a parameter of the applied treatment for effective or safe application of the treatment in accordance with a detected feature or characteristic. The controller may automatically activate or deactivate application of the skin treatment (e.g., delivering energy into the skin) based on sensed displacement, detected skin characteristics (e.g., skin tone, redness, texture, wrinkles, or other characteristics), on a measured time (e.g., duration of treatment or rate of displacement), or on any combination of the above.

In accordance with some embodiments of the present invention, components of an optical displacement sensor may be utilized to sense or measure proximity of the skin surface. For example, optics of the optical displacement sensor may be configured to produce a focused image of the skin surface when the treatment head is placed on the skin surface. An acquired image may be analyzed to determine if a feature of the skin surface is in focus (e.g., by detecting, measuring, or calculating a sharpness of an edge in the image). A deviation from focus (e.g., determined from edge blurring) may be indicative of a distance between the optical displacement sensor and the skin surface. As another example, in some cases, a reflectance of the skin surface, or a feature of the skin surface, may be known at least approximately. In such a case, a measured or detected brightness of an image of the skin surface or feature may be indicative of a distance between the optical displacement sensor and the skin surface

Phototherapy devices may produce high-intensity light. Such light could be to the eyes of a user (e.g., operator or person being treated) when activated to produce light while removed from the skin surface. In accordance with some embodiments of the present invention, the controller may be configured to utilized components of an optical displacement sensor to determine whether the treatment head is within a predetermined distance from the skin surface. Production of the light may be discontinued when the distance from the skin surface exceeds the predetermined distance.

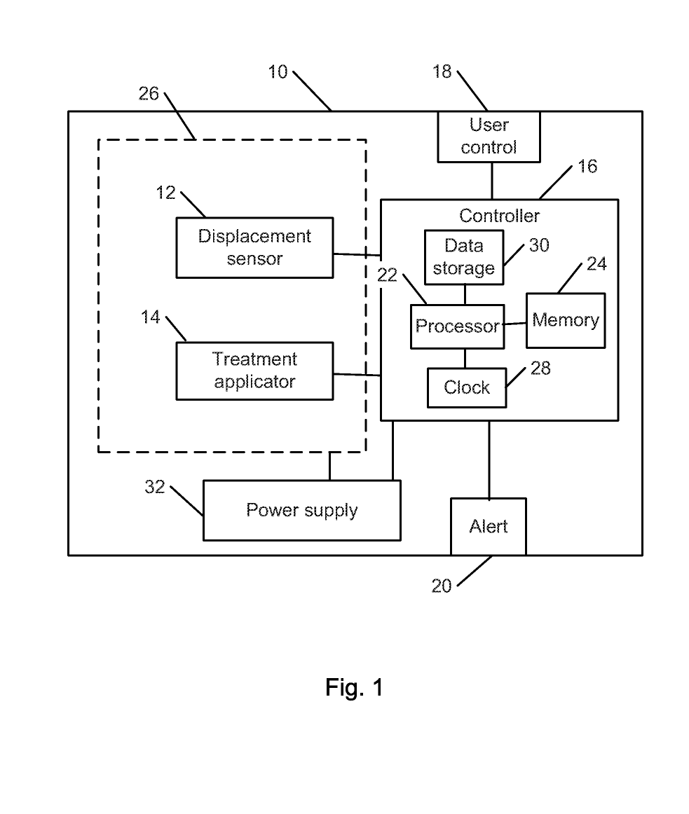

FIG. 1 is a schematic illustration of a skin treatment device, in accordance with an embodiment of the present invention.

Skin treatment device 10 includes a treatment head 26. Treatment head 26 is configured to be placed on a skin surface when treatment is being applied to that skin surface. Treatment head 26 includes at least treatment applicator 14 and displacement sensor 12. In some cases, treatment head 26 may include additional sensors or components. In some cases, treatment head 26 may be detachable and re-attachable to the remainder of skin device 10.

Treatment head 26 includes a treatment applicator 14. For example, treatment applicator 14 may include a radiation source (e.g., of visible, ultraviolet, infrared, or radiofrequency radiation), an electromagnetic device, a heat source, a mechanical device (e.g., vibrator, mechanical massager, shaver, or other mechanical device), or another skin treatment device. Treatment applicator 14 may include a system to deliver a substance from a reservoir or container of the substance to the skin (e.g., topical or subcutaneous application). Operation of treatment applicator 14 is controlled by controller 16. Controller 16 may control operation of treatment applicator 14 in accordance with user input via operation of user control 18, in accordance with sensed information from displacement sensor 12, in accordance with programmed instructions, or in accordance with any combination of the above.

Treatment head 26 includes displacement sensor 12. Displacement sensor 12 includes one or more displacement sensing devices that are configured to sense a displacement of treatment head 26 relative to a skin surface. For example, displacement sensor 12 may operate as a mechanical or optical displacement sensor.

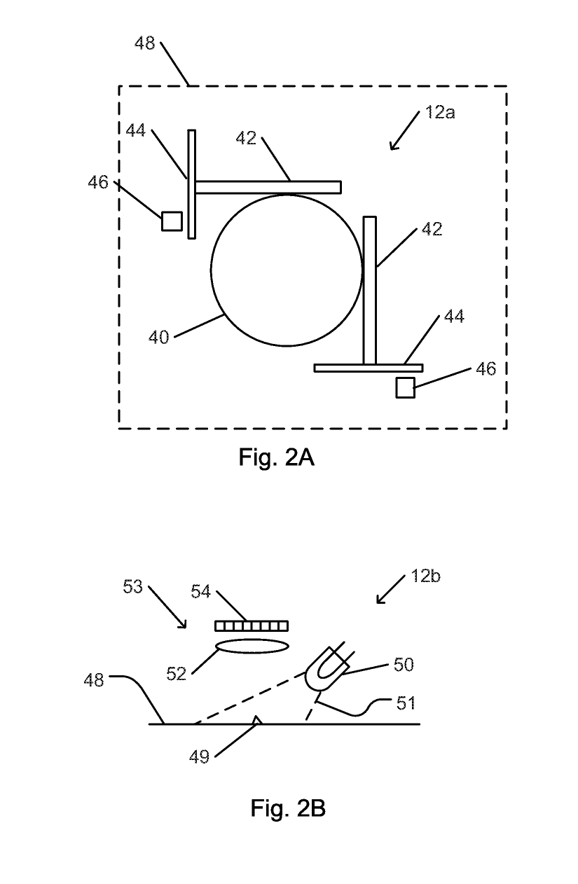

FIG. 2A is a schematic illustration of a mechanical displacement sensor for a skin treatment device, in accordance with an embodiment of the present invention.

Mechanical displacement sensor 12a includes a ball 40 that is configured to remain in contact with (e.g., remain tangent to) skin surface 48 (shown as behind ball 40). When ball 40 is in contact with skin surface 48 and mechanical displacement sensor 12a is displaced laterally relative to skin surface 48, ball 40 may roll. The two-dimensional angular displacement or rotation of ball 40 may thus be related to the two-dimensional linear displacement of mechanical displacement sensor 12a relative to skin surface 48.

Rollers 42 are configured to maintain contact with ball 40 as ball 40 rolls. The longitudinal axes of rotation of different rollers 42 are not parallel to each other (e.g., are orthogonal to each other). Each roller 42 is configured to roll about its longitudinal axis by an angular displacement (rotation angle) that is related to the rotation of ball 40. Coaxially attached to each roller 42 is an encoder disk 44 (e.g., in the form of a chopper wheel) that rotates together with the attached roller 42. An encoder reader device 46 is configured to produce an electrical signal that is indicative of the rotation of encoder disk 44. A processor (e.g., processor 22 of controller 16 or another processor) is configured to interpret the produced electrical signal to determine a rotation of roller 42. The rotation of roller 42 is indicative of a rotation of ball 40 in one dimension, and of a displacement in one dimension of mechanical displacement sensor 12a relative to skin surface 48. The processor may thus be configured to interpret the produced electrical signals as a two-dimensional displacement of mechanical displacement sensor 12a relative to skin surface 48.

FIG. 2B is a schematic illustration of an optical displacement sensor for a skin treatment device, in accordance with an embodiment of the present invention.

Optical displacement sensor 12b includes a light source 50 (e.g., LED, laser, xenon lamp, or other light source). Light source 50 produces illumination 51 that illuminates a region of skin surface 48. Light source 50 may include two or more different component light producing units, each emitting light with different characteristics. For example, light source 50 may include two or more different LED or laser sources with each emitting light at a different wavelength. A particular component light producing unit may be selected by a user or automatically, e.g., in accordance with one or more characteristics of skin surface 48.

Camera 53 is configured to acquire sequential images of all or part of the illuminated region. For example, camera 53 may include optics 52 and sensor array 54. For example, optics 52 may include one or more of a lens (or set or array of lenses), a mirror (or a set or array of mirrors), aperture, collimator, iris, filter, or other optical component. The optics may collect light from the illuminated region and to focus the light on sensor array 54. Sensor array 54 may include charge-coupled devices, photodiodes, or other light sensing components.

The illuminated region may include one or more skin features 49 that are distinguishable in an acquired image from surrounding areas of the illuminated region. For example, an image of skin feature 49 may be distinguished from an image of a surrounding area by its brightness, color, or texture (e.g., patterning). A processor (e.g., processor 22 of controller 16 or another processor) is configured to analyze successively acquired images (or frames). Analysis of the images may detect that an image of skin feature 49 has moved from one frame to another. The processor is configured to interpret such movement as a two-dimensional displacement of optical displacement sensor 12b relative to skin surface 48.

A displacement sensing device may be based on other optical (e.g., rangefinder or image processing technology), mechanical (e.g., incorporating sliding or extendible mechanical elements), acoustic (e.g., ultrasonic range-finding), electromagnetic (e.g., incorporating proximity sensor technology), or other principles or technologies.

Controller 16 is configured to control operation of components of skin treatment device 10. For example, controller 16 may be configured to control operation of displacement sensor 12, of treatment applicator 14, of alert device 20, or of any combination of the above.

Controller 16 includes processor 22. Processor 22 may include one or more intercommunicating processing units. For example, a processing unit of processor 22 may include a processing unit that is associated displacement sensor 12 or with treatment applicator 14. Processor 22 may be configured to operate in accordance with programmed instructions stored in memory 24 or in data storage device 30.

Processor 22 may communicate with clock 28. Clock 28 may be configured to produce a signal that is indicative of an absolute time (e.g., corresponding to a time in accordance with a standard clock) or of a relative time. For example, a relative time may include time elapsed from as event, such as from powering on of skin treatment device 10, or from beginning of operation of a component of skin treatment device 10 (e.g., controller 16, displacement sensor 12, treatment applicator 14, user control 18, or another component of skin treatment device 10). Processor 22 may be configured to utilize a time signal that is produced by clock 28. For example, the time signal may be utilized in calculating a rate of displacement, a rate or duration of application of treatment, or another rate or duration.

Processor 22 may communicate with memory 24. Memory 24 may include one or more volatile or nonvolatile memory devices. Memory 24 may be utilized to store, for example, programmed instructions for operation of processor 22, data or parameters for use by processor 22 during operation, or results of operation of processor 22. For example, memory 24 may be used to store displacement-related information (e.g., a position or displacement relative to a starting point, time of sensing of that displacement, duration or type of treatment applied at that position, or other displacement-related information). The stored displacement-related information may be analyzed to yield a calculated displacement rate, whether further treatment is to be applied at a given position, or other results of analysis of the displacement-related information.

Processor 22 may communicate with data storage device 30. Data storage device 30 may include one or more fixed or removable nonvolatile data storage devices. For example, data storage device 30 may include a computer readable medium for storing program instructions for operation of processor 22. It is noted that a component of storage device 20 may be remote from processor 22. In such cases storage device 20 may be a storage device of a remote server storing programmed instructions in the form of an installation package or packages that can be downloaded and installed for execution by processor 22. Data storage device 30 may be utilized to store data or parameters for use by processor 22 during operation, or results of operation of processor 22. For example, data storage device 30 may be utilized to store treatment parameters, displacement-related information, or other information related to operation of skin treatment device 10.

Skin treatment device 10 may include user control 18. User control 18 may include one or more components that may be operated by a user of skin treatment device 10. For example, user control 18 may include one or more switches, pushbuttons, levers, knobs, touch screens, keys, pointing devices, or other components that may be operated by a user of skin treatment device 10. User control 18 may include a proximity or contact sensor (e.g., optical, acoustic, mechanical, electromagnetic, or other proximity or contact sensor) that senses when treatment head 26 is placed near or in contact with a skin surface. Operation of aspects of controller 16 may be controlled or determined by operation of user control 18. Such aspects may include, for example, powering on or off of skin treatment device 10, operation of displacement sensor 12, operation of treatment applicator 14, operation of alter device 20, to enter information related to a skin surface being treated, or other aspects of operation operate of skin treatment device 10.

Skin treatment device 10 may include an alert device 20. Alert device 20 may be operated by controller 16. Alert device 20 may include one or more components that are capable of producing visible, audible, or tactile indications or signals. For example, alert device 20 may include one or more lights, displays, or other devices that may produce visible signals. Alert device 20 may include one or more bells, buzzers, tone generators, speakers, or other devices that may produce audible signals. Alert device 20 may include one or more vibrators or other devices that may produce tactile or palpable signals. Alert device 20 may be operated by processor 22 to indicate one or more situations. For example, alert device 20 may be operated by processor 22 to indicate an operational status of skin treatment device 10. Such a status may include, e.g., power on or off, treatment being applied or not being applied, displacement rate adequate or not adequate, treatment head 26 positioned on region already treated, sufficient or insufficient power for operation, or another operational status. Alert device 20 may be operated by processor 22 to notify a user of skin treatment device 10 of an action that is to be performed by the user or to request that the user perform the action. Such actions may include, e.g., operation of a user control 18, changing a displacement rate, moving treatment head 26, providing adequate power (e.g., recharge or replace a battery of power supply 32, connect to an external power source, or otherwise provide power), or another action.

Skin treatment device 10 includes power supply 32. For example, if skin treatment device 10 is a self-contained device, power supply 32 may include a rechargeable or replaceable battery. Power supply 32 may include a power generating component, such as a photovoltaic cell or array for converting ambient light to electrical power. Power supply 32 may include a connection to an external power supply, such as to a power mains, transformer, converter, generator, charging unit, or other external power supply. Power supply 32 may include one or more fuses or circuit breakers, transformers, current or voltage regulators, or other components for enabling safe and proper operation of components of skin treatment device 10. Power supply 32 may provide electrical power for operation or one or more of displacement sensor 12, treatment applicator 14, controller 16, alert device 20, user control 18, or other component of skin treatment device 10.

Processor 22 may be configured to execute a method of operation of skin treatment device 10.

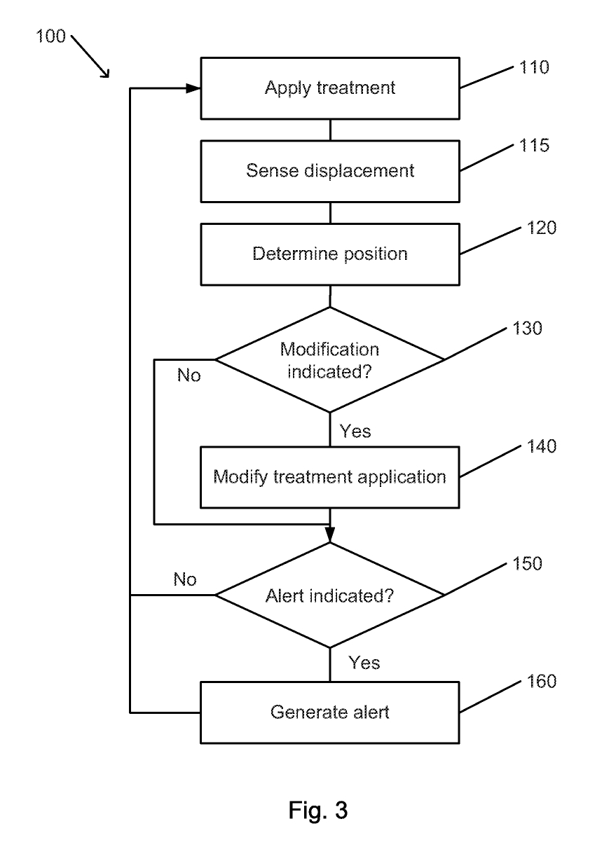

FIG. 3 is a flowchart of a method of operation of a skin treatment device in accordance with an embodiment of the present invention.

It should be understood with respect to any flowchart referenced herein that the division of the illustrated method into discrete operations represented by blocks of the flowchart has been selected for convenience and clarity only. Alternative division of the illustrated method into discrete operations is possible with equivalent results. Such alternative division of the illustrated method into discrete operations should be understood as representing other embodiments of the illustrated method.

Similarly, it should be understood that, unless indicated otherwise, the illustrated order of execution of the operations represented by blocks of any flowchart referenced herein has been selected for convenience and clarity only. Operations of the illustrated method may be executed in an alternative order, or concurrently, with equivalent results. Such reordering of operations of the illustrated method should be understood as representing other embodiments of the illustrated method.

Treatment device operation method 100 may be executed by a processor of a controller of a skin treatment device. Treatment device operation method 100 may be executed automatically when the skin treatment device is being operated (e.g., powered on, treatment head placed on or near the skin surface, or when treatment application is enabled, e.g., by operation of a suitable user control).

The skin treatment device may be operated to apply a treatment to the skin surface (block 110). For example, application of treatment may include deposition of energy (e.g., electromagnetic radiation, heat, mechanical, or other energy) or other application to a treated region of the skin.

During operation of the skin treatment device, a displacement of the treatment head of the device relative to the skin surface may be sensed (block 115). For example, a displacement sensor of the device may automatically operate to determine a displacement of current position of the treatment head relative to a previous or initial position of the treatment head.

The sense displacement may be used to calculate a current position of the treatment head (block 120). The sensed displacement may be vectorially added to a previously determined position to yield a current position of the treatment head. For example, an initial position may be set when the device is powered on, when the treatment head is first placed on or near the skin surface, or when application of treatment is enabled. Each sensed displacement may be vectorially added to the initial position to yield a position of the treatment head after each displacement. In some cases (e.g., with an optical displacement sensor) rotation of the treatment head may also be sensed to yield a current relative orientation of the treatment head. A region of the skin surface to which treatment is being applied may be inferred from the sensed position (and orientation, if known). A sequence of sensed positions may be stored in a memory that is associated with the device controller.

The sensed displacement together with clock data may be used to determine a current rate of displacement (vector velocity) of the treatment head relative to the skin surface.

It may be determined that modification of application of the treatment is indicated (block 130). For example, it may be determined that a current position of the treatment head partially or completely covers a region to which treatment had been previously applied during the current treatment application session. As another example, it may be determined that the treatment head has been moved from a previously treated position to a position that has not yet been treated. Modification of treatment may be otherwise indicated. For example, analysis of an image of the skin surface (e.g., as acquired by an optical displacement sensor) may indicate no need to apply treatment to the currently covered region, or may indicate another modification of the applied treatment. Such analysis may indicate, e.g., that a condition to be treated is not present or is otherwise different at the current position of the treatment head, or that the skin surface is otherwise different at the current position from the surface at previously treated positions. It may be determined rate that application of the treatment is to be adjusted to correspond to a determined rate of displacement of the treatment head.

When no modification is indicated, execution of treatment device operation method 100 may continue (e.g., to block 150 or return to block 110).

When modification of treatment application is indicated, application of the treatment may be modified (block 140). For example, application of the treatment may be interrupted or pause, may be resumed (e.g., if previously interrupted or paused), or may be otherwise modified (e.g., increase or decrease intensity of applied radiation or other treatment, or other modification). Treatment may continue to be applied, or not applied, in accordance with the modification until another modification is indicated.

Generation of an alert may be indicated (block 150). It may be determined that a user of the skin treatment device is to be informed or alerted regarding a operational status or change in operation of the skin treatment device. For example, generation of an alert may be indicated when the device has interrupted application of the treatment, resumed or started application of the treatment, or has modified application of the treatment. An alert may be indicative of a result of a comparison of a calculated current rate of displacement with a correct rate of displacement. For example, generation of an alert may be indicated when a calculated rate of displacement (e.g., caused by too rapid or too slow manipulation of the skin treatment device by the user) of the treatment head deviates from a correct (e.g., required, desirable, or recommended) range of displacement rates. Generation of an alert may be indicated when user attention is recommended. For example, user attention may be recommended to perform maintenance on the skin treatment device (e.g., cleaning, battery charging or replacement, connection to external power source, part replacement, refilling or replacement of a container or reservoir of an applied substance, or other maintenance).

When no generation of an alert is indicated, operation of the skin treatment device may continue (return to block 110).

When generation of an alert is indicated, an alert is generated (block 160). For example, the alert may be visible (e.g., turning an indicator light on or off, flashing or blinking an indicator light, displaying appropriate graphic or alphanumeric content, or otherwise visible), audible (e.g., generate a sound or sequence of sounds, synthesize or play a recording of a message in a spoken language, or otherwise audible), palpable (e.g., vibrating or knocking), or otherwise detectable by a human user of the device.

After generation of the alert, operation of the skin treatment device may continue (return to block 110).

1. A device for treating a skin surface, the device comprising:
a treatment applicator to apply a treatment to a region of the skin surface on which the treatment applicator is placed;
a displacement sensor to sense a displacement of the device relative to the skin surface when the treatment applicator is placed on the skin surface; and
a controller to control application of the treatment in accordance with the sensed displacement.

2. The device of claim 1, wherein the treatment applicator comprises a radiation source to irradiate the region.

3. The device of claim 2, wherein the radiation source comprises a light emitting diode, a laser, or a xenon lamp.

4. The device of claim 2, wherein an intensity of the radiation is controllable in accordance with the displacement.

5. The device of claim 1, wherein the controller is configured to control application of the treatment avoid excessive application of the treatment to a single region of the skin surface.

6. The device of claim 1, further comprising an alert device.

7. The device of claim 6, wherein the controller is configured to control the alert device to indicate a comparison of a rate of the sensed displacement with a correct rate of displacement.

8. The device of any of claim 1, wherein the device is handheld and self-contained.

9. The device of any of claim 1, wherein the displacement sensor comprises an optical sensor that is configured to acquire a sequence of images of the skin surface.

10. The device of claim 1, wherein the displacement sensor comprises a ball that is configured to rotate when placed on the skin surface and displaced along the skin surface.

11. A method to control a device for treating a skin surface, the method comprising:
obtaining a sensed displacement of the treatment head relative to the skin surface when the treatment head is placed on the skin surface; and
controlling operation of a treatment applicator to apply a treatment to the skin in accordance with the sensed displacement.

12. The method of claim 11, wherein controlling the operation comprises calculating a current position of the treatment head on the skin surface.

13. The method of claim 12, wherein controlling the operation comprises modifying application of the treatment when the current position is within a predetermined distance from a position at which the treatment was previously applied.

14. The method of claim 11, wherein controlling the operation comprises calculating a rate of displacement of the treatment head.

15. The method of claim 14, wherein controlling the operation comprises adjusting application of the treatment to correspond to the calculated rate of displacement.

16. The method of claim 14, further comprising comparing the calculated rate of displacement with a correct rate of displacement.

17. The method of claim 16, further comprising generating an alert indicative of the result of the comparing of the calculated rate with the correct rate.

18. The method of claim 11, further comprising sensing a characteristic of the skin surface and controlling operation of the treatment applicator to apply the treatment in accordance with the sensed characteristic.

19. The method of claim 11, further comprising sensing a proximity of the skin surface and controlling operation of the treatment applicator to apply the treatment in accordance with the sensed proximity.

20. The method of claim 11, wherein application of the treatment comprises irradiating the skin surface.
